(19) United States
(12) Patent Application Publication
(10) Pub. No.: US 2009/0304711 A1
(43) Pub. Date: Dec. 10, 2009

(54) Combinatorial Therapy of Cancer and Infectious Diseases with Anti-B7-H1 Antibodies

(76) Inventors: Drew Pardoll, Baltimore, MD (US); Lieping Chen, Baltimore, MD (US); Charles Drake, Baltimore, MD (US); Andrea Cox, Baltimore, MD (US)

Correspondence Address: KING & SPALDING, 1180 PEACHTREE STREET , NE, ATLANTA, GA, 30309-3521, US

(21) Appl. No.: 12/441,996

(22) PCT Filed: Sep. 20, 2007
(86) PCT No.: PCT/US07/79058

§ 371 Date: Aug. 12, 2009

Related U.S. Application Data
(60) Provisional application No. 60/846,031, filed on Sep. 20, 2006.

(51) Int. Cl. A61K 39/395; A61K 39/00

(52) U.S. Cl. 424/158.1; 424/184.1

(57) ABSTRACT

Described are uses of an agent that reduces B7-H1 interaction with PD-1, and particularly monoclonal antibodies that bind to B7-H1 and interfere with B7-H1 interaction with PD-1 in combination with a vaccine to provide synergistic effects. The application provides methods of treatment and vaccination based on the combination of these compounds on T cell responses.

### CROSS-REFERENCE TO RELATED APPLICATIONS

This application claims priority to U.S. Provisional Application No. 60/846,031, filed Sep. 20, 2006, the disclosure of which is incorporated herein by reference.

### FIELD OF THE INVENTION

This application relates to uses of antibodies that block interaction between B7-H1 and PD-1 to enhance immune responses by reducing T-cell mediated tolerance to antigenic stimuli. The application provides methods of treatment and vaccination based on the effects of the antibodies on T cell responses.

### BACKGROUND

Immune modulation is a critical aspect of the treatment of a number of diseases and disorders. T cells in particular play a vital role in fighting infections and have the capability to recognize and destroy cancer cells. Enhancing T cell mediated responses is a key component to enhancing responses to therapeutic agents.

Immunotherapy is currently a major focus of cancer therapy, wherein therapeutic cancer vaccines may represent major alternatives and/or adjuvant therapies besides chemotherapy. It is now well established that tumor-specific and tumor-associated antigens derive from the genetic and epigenetic alterations that underlie all cancers. Genetic instability in cancers is a consequence of deletion or mutational inactivation of genome guardians, such as p53. The genetic instability of cancer cells means that new antigens are constantly being generated in tumors as they develop and progress. The accumulation of karyotypic abnormalities in advanced undifferentiated cancers emphasizes the level of genetic instability in tumors. This genetic instability does not occur in normal non-transformed tissues which maintain their genome guardians and therefore a stable biochemical and antigenic profile. In addition to the thousands of mutational events that occur during tumorigenesis, hundreds of genes that are either inactive or expressed at relatively low levels in the normal tissue counterparts, are up-regulated significantly in cancers. Although these epigenetic changes do not formally create tumor-specific neoantigens, they raise the concentration on encoded proteins dramatically or turn on genes normally silent in non-transformed adult tissues. Epigenetic alterations thus effect the antigenic profile of the tumor cell as much as genetic alterations.

These genetic and epigenetic alterations in cancer cells generate tumor-associated antigens of two distinct types: 1) Unique tumor specific antigens that are the products of mutation and 2) Tumor selective antigens expressed at much higher levels in tumors than normal tissues. Tissue specific differentiation antigens represent another category of target tumor associated antigen applicable for cancers derived from dispensable tissues such as melanoma and prostate cancer. Finally, viral antigens expressed by virus-induced cancers as cervical cancer (HPV), hepatoma (HBV, HCV), Hodgkins lymphoma (EBV) and nasopharyngeal carcinoma (EBV) represent excellent targets for antigen-specific immunotherapy. In particular, chronic viral diseases, such as HBV and HCV, can be identified prior to development of cancer and can be eliminated with appropriate immune intervention.

The adaptive immune system provides tremendous potential as a weapon against cancer via its capacity to target tumor-associated antigens. First and foremost, the genetic diversity mechanisms for B cells and T cells (via their T cell receptor) confers the capacity to generate roughly 1022 different immunoglobulins and 1018 different T cell receptors, respectively. Both antibodies and T cell receptors can distinguish biochemical moieties that differ by as little as a single methyl group. Therefore, the combination of antibodies and T cells offers the ability to recognize even subtle biochemical differences that are either specific or selective to tumor cells relative to there normal counterparts. T cells additionally offer the capacity to recognize intracellular antigens in essentially any cellular compartment. This is because T cells, via their T cell receptor, recognize peptide-MHC complexes on the cell surface. The majority of peptides presented by MHC molecules on the cell surface are originally derived from processing of proteins in intracellular compartments. Following loading, the peptide-MHC complexes are transported to the cell surface for recognition by T cells. Therefore, the MHC system represents a conveyer belt bringing pieces of intracellular antigens to the surface for recognition by T cells.

However, although cancer cells frequently express tumor antigens that, in principle, can be recognized by the patient's immune system, resultant immune responses are ineffective and often do not parallel clinical tumor regression. A number of genetically modified vaccines including idiotypic vaccines for lymphoma and GM-CSF transduced vaccines for multiple cancer types as well as recombinant viral and bacterial vaccines are demonstrating promising activity in Phase I/II trials. Essentially all tumor vaccines work through the activation of tumor-specific T cell responses. However, there is an emerging consensus that even the most potent therapeutic vaccines provide limited activity. No therapeutic vaccine for either cancer or a chronic infectious disease has been successful in Phase III trials to date. The scientific potential for enhancing the limited activity of cancer vaccines rests with the multiple immune regulatory pathways that either amplify or down-modulate antigen driven immune responses.

This raises an essential question in tumor immunology: Why are neoplasms expressing tumor antigens not eliminated by the patient's own immune system? At the fundamental level, three elements determine T cell responsiveness to an antigen:

1. Signal 1. The first element, termed “signal one”, is transmitted by the T cell receptor which acts as a signal transducer for external stimuli to initiate T cell activation. Small peptide fragments derived from proteolysis of antigens are presented to the T cell receptor by MHC (HLA in humans) molecules expressed by antigen presenting cells. The critical antigen-presenting cell that activates T cell responses is the dendritic cell. Therefore, it is now appreciated that essentially all vaccines stimulate immune responses through transfer of antigen to dendritic cells, which in turn degrade it into peptides and present those peptides on MHC molecules to the T cell via TCR recognition.

2. Signal 2. When T cells receive Signal 1 through TCR engagement without additional signals, they enter an unresponsive, or anergic state, in which they do not mediate effector function. This represents one mechanism for self tolerance that protects normal tissues from immune destruction and probably also represents a mechanism by which tumor specific T cells in patients are naturally unresponsive to there tumor, thereby allowing it to grow. The critical second element in T cell activation—collectively referred to as “Signal 2”—is delivered by a large number of costimulatory molecules expressed by the antigen presenting cell which interact with costimulatory receptors on the T cell. The prototypical costimulatory molecules are B7.1 and its homologue B7.2. B7.1/7.2 costimulate T cells by interacting with the CD28 receptor on T cells. Signals delivered by both the T cell receptor (Signal 1) and CD28 collaborate to enhance T cell activation. Six additional B7 family members have been identified over the last five years—B7RP-1 (also called ICOS-L, B7h, B7-H2), B7-H1 (also called PD-L1), B7-DC (also called PD-L2), B7-H3, B7-H4 (also called B7s, B7x) and B7-H5. Most of these possess additional costimulatory functions and in some cases can collaborate with B7.112 to costimulate T cells through receptors distinct from CD28.
3. Immunologic checkpoints. The final element in T cell regulation is represented by inhibitory pathways, termed “immunologic checkpoints”. There are many immunologic checkpoints that serve two purposes. One is to help generate and maintain self-tolerance among T cells specific for self-antigens. The other is to restrain the amplitude of normal T cell responses so that they do not “overshoot” in their natural response to foreign pathogens. Two of the more recently discovered B7 family members—B7-H1 and B7-DC, also appear to interact with costimulatory and counter-regulatory inhibitory receptors. PD-1, which is upregulated on T cells upon activation, appears to be a counter-regulatory immunologic checkpoint, especially when it binds either B7-DC or B7-H1 (see e.g. Iwai, et al. (2005) Int. Immunol. 17:133-44).

In addition to anergy that occurs is cells are exposed to Signal 1 without Signal 2, recently it has become clear that regulatory T cells play an important role in maintaining tolerance. Regulatory T cells suppress auto-reactive T cells. Thus, as the level of regulatory T cells decreases, the potential for autoimmunity rises. Interestingly, tumors have been shown to evade immune destruction by impeding T cell activation through inhibition of co-stimmulatory factors in the B7-CD28 and TNF families, as well as by attracting regulatory T cells, which inhibit anti-tumor T cell responses (see Wang (2006) Immune Suppression by Tumor Specific CD4⁺ Regulatory T cells in Cancer. Semin. Cancer. Biol. 16:73-79; Greenwald, et al. (2005) The B7 Family Revisited. Ann. Rev. Immunol. 23:515-48; Watts (2005) TNF/TNFR Family Members in Co-stimulation of T Cell Responses Ann. Rev. Immunol. 23:23-68; Sadum, et al. (2007) Immune Signatures of Murine and Human Cancers Reveal Unique Mechanisms of Tumor Escape and New Targets for Cancer Immunotherapy. Clin. Canc. Res. 13(13): 4016-4025).

As engineered cancer vaccines continue to improve, it is becoming clear that two of the major barriers to their ability to induce therapeutic anti-tumor responses are the activation of immunologic checkpoints that attenuate T cell dependent immune responses, both at the level of initiation and effector function within tumor metastases.

One immunologic checkpoint that operates at the level of effector T cell responses within tumors is B7-H1. B7-H1 encompasses a recently discovered cell surface glycoprotein within the B7 family of T-cell co-regulatory molecules. Recent studies reveal that B7-H1 possesses dual functions of co-stimulation of naive T cells and inhibition of activated effector T cells. The aberrant cellular expression and deregulated function of B7-H1 have been reported during chronic viral and intracellular bacterial infection, as well as in many autoimmune diseases and cancers.

It has been shown that B7-H1 is expressed on certain tumors and can be upregulated upon exposure to interferon-gamma and can inhibit antitumor immune responses. In addition, some human tumors acquire the ability to aberrantly express B7-H1. It has been suggested that B7-H1/PD-1 interactions negatively regulate T cell effector functions and have a role in tumor evasion (see Blank et al. (2006) Int. J Cancer. 119:317-27; Curiel, et al. (2003) Nat. Med. 9:562-67; Hirano, et al. (2005) Cancer Res. 65:1089-96). Tumor-associated B7-H1, as well as B7-H1 on activated lymphocytes, has been shown to impair antigen-specific T-cell function and survival in vitro. Transduction of B7-H1-tumors with the B7-H1 gene results in surface expression of B7-H1 with resultant protection from elimination by a tumor vaccine. B7-H1 has also been implicated in regulating T cells in other disorders (see eg. Das, et al. (2006) J. Immunol. 176:3000-9). Consequently, tumor-associated B7-H1 has garnered much attention in the recent literature as a potential inhibitor of host antitumoral immunity (see e.g. Thompson, et al. (2005) Cancer 104:2084-91).

Hirano et al. describe the effects of blockade of B7-H1 and PD-1 by monoclonal antibodies, however fail to provide methods by which efficacy of vaccination can be enhanced.

U.S. Pat. No. 7,029,674 to Wyeth, discloses methods for down-modulating an immune response comprising contacting an immune cell with an agent that modulates the interaction between PD-1 and a PD-1 ligand (e.g., soluble forms of PD-1 or PD-1 ligand or antibodies to PD-1) to thereby modulate the immune response. In some embodiments, the agent can be a monovalent antibody that binds to PD-1.

U.S. Application No. 2003/0039653 to Chen and Strome describes methods of enhancing responsiveness of a T cell involving interfering in the interaction between a T cell and a B7-11 molecule.

U.S. Application No. 2006/0083744 to Chen et al. describes methods of diagnosis by assessing B7-H1 expression in a tissue from a subject that has, or is suspected of having, cancer, methods of treatment with agents that interfere with B7-H1-receptor interactions, methods of selecting candidate subjects likely to benefit from cancer immunotherapy and methods of inhibiting expression of B7-H1.

There remains a need for therapies that provide enhancement of the efficacy of therapeutic vaccines, particularly for treatment and prevention of abnormal cell proliferation and for treatment of infectious diseases and disorders.

It is an object of the present invention to provide methods of treatment that enhance the efficacy of vaccines and reduce T cell anergy in certain disease states. It is a specific object of the invention to provide improved methods of preventing or treating abnormal cell proliferation and infectious diseases in a host.

### SUMMARY

It has been found that a combination of 1) an agent that blocks B7-H1 interactions with its ligand PD-1 and 2) a vaccine is synergistic in overcoming natural T cell tolerance or functional inactivation induced by tumor cells or by chronic infections. Therefore, in one embodiment, a method of enhancing efficacy of a vaccine is provided comprising administering an agent that blocks B7-H1 interactions with PD-1 in combination with the vaccine to a host in need thereof. In certain embodiments, a method of treating or preventing abnormal cell proliferation in a host is provided, comprising administering an agent that blocks B7-H1 interactions with PD-1 in combination with a vaccine against the cancer to a host in need thereof. In certain other embodiments, a method of treating chronic infection in a host is provided comprising administering an agent that blocks B7-H1 interactions with PD-1 in combination with a vaccine against the infection to a host in need thereof.

In one embodiment, the agent that blocks B7-H1 binding to PD-1 is an antibody. In certain embodiments, the agent is an antibody that binds to B7-H1 and inhibits its interaction with PD-1. In certain embodiments, the agent is an agent that binds to B7-H1 and changes its conformation so that the protein no longer binds to PD-1. In other embodiments, the agent binds to B7-H1 at the PD-1 binding site and blocks interaction with PD-1. In some other embodiments, the agent binds to PD-1 and blocks PD-1 from interaction with B7-H1. In certain embodiments, the agent is an antibody to PD-1.

In some embodiments, the agent and vaccine can be administered in the same composition. In certain other embodiments, the agent and vaccine are administered in separate compositions. In some embodiments, the agent and vaccine are administered concurrently in separate preparations. In other embodiments, the agent is administered before administration of the vaccine. In certain embodiments, the agent is administered within one hour of the vaccine. In certain embodiments, the administration of the agent and vaccine are overlapping but not contiguous. For example, in certain embodiments, the vaccine can be administered intravenously for at least one hour and the agent may be administered orally during the intravenous administration.

In one embodiment, a composition comprising an agent that blocks B7-H1 binding to PD-1 and a vaccine is provided. In certain embodiments, the agent is an antibody and in certain specific embodiments, is an antibody that binds B7-H1. In some embodiments, the anti-B7-H1 antibody binds to the protein and changes its conformation so that B7-H1 no longer binds to PD-1.

In some embodiments, the composition is an injectible composition. In certain embodiments, the composition comprises a carrier suitable for intravenous administration. In certain other embodiments, the composition comprises a carrier suitable for subcutaneous or intramuscular injection. In certain other embodiments, the composition comprises a carrier suitable for intraperitoneal administration. In other embodiment, the composition can be administered by oral administration.

In another embodiment, a method of eliciting an immune response in a host is provided comprising administering an agent that interferes with B7-H1 binding to PD-1 in combination with an antigen to the host. In certain embodiments, the host is suffering from an infection. In one subembodiment, the infection is a chronic infection. In another subembodiment, the infection is an acute infection. In one embodiment, the infection is due to a virus. In another embodiment, the infection is due to a bacteria. In one embodiment, the infection is a chronic infection such as HIV, HBV, EBV, HPV or HCV.

In one embodiment, the antigen is a viral protein. In another embodiment, the antigen is a bacterial protein. In yet another embodiment, the antigen is a mammalian protein. In certain embodiments, the antigen is expressed in a Listeria species. The Listeria species can be a Listeria monocytogenes. Methods of producing Listeria vaccines, including Listeria species expressing antigens of interest are discussed in U.S. Patent Application Publication Nos. 2004/0228877, 2005/0249748 and 2005/0281783. In certain embodiments, the Listeria species is attenuated for entry into non-phagocytic cells as compared to a wild type Listeria species. In certain cases, the Listeria species is one in which the inlB gene has been deleted (i.e., a strain attenuated for entry into non-phagocytic cells, for example, hepatocytes via the c-met receptor) or both the actA gene and the inlB genes have been deleted (i.e., a strain attenuated for both entry into non-phagocytic cells and cell-to-cell spread).

In separate principal embodiments, methods of treating or preventing abnormal cell proliferation in a host are provided. These methods can reduce the risk of developing cancer in the host. In other embodiments, the methods reduce the amount of cancer in a host. In yet other embodiments, the methods reduce the metastatic potential of a cancer in a host. The methods can also reduce the size of a cancer in a host.

In some embodiments, administration of the agent reduces tolerance of T cells to a cancer. In these embodiments, the agent that reduces B7-H1 interaction with PD-1 increases susceptibility of cancer cells to immune rejection. In certain embodiments, the immune response elicited by the agent that reduces B7-H1 interaction with PD-1 is a reduction in regulatory T cells. In yet other embodiments, the agent inhibit generation, expansion or stimulation of regulatory T cells. In further embodiments, the agent causes a reduction in T cell anergy. The reduction in T cell anergy can be in tumor-specific T cells.

In one specific embodiment, a method of treating or preventing abnormal cell proliferation in a host is provided comprising administering to a host in need thereof an agent that reduces B7-H1 interaction with PD-1 in combination or alternation with a mammalian cell based vaccine.

In one embodiment, the mammalian cell based vaccine is a whole mammalian cell. In certain embodiments, the vaccine is a tumor cell that is not actively dividing. The tumor cell can be irradiated. In certain embodiments, the cell is genetically modified. In some embodiments, the cell can be secreting an activation factor for an antigen-presenting cell. In certain embodiments, the cell secretes, for example constitutively secretes, a colony stimulating factor and can specifically secrete a granulocyte-macrophage colony stimulating factor (GM-CSF). In some embodiments, the vaccine is viral cell based vaccine. In other embodiments, the vaccine is not based on a cell. In certain embodiments, the vaccine is a DNA-based vaccine. In other embodiments, the vaccine is not a DNA based vaccine.

In certain embodiments, the vaccine is an antigen specific vaccines such as recombinant viral vaccines, recombinant bacterial vaccines, recombinant protein based vaccines or peptide vaccine. A recombinant vaccine incorporates either tumor specific antigens or antigens derived from chronic viruses such as HCV, HBV, HIV, EBV or HPV.

In one embodiment, the agent that reduces B7-H1 interaction with PD-1 reduces tolerance of T cells to a cell in the cell based vaccine. In this embodiment, the agent increases susceptibility of tumor cells to immune rejection. In one embodiment, the immune response is a reduction in regulatory T cells. In one embodiment, the agent enhances generation of memory T cells. In yet another embodiment, the agent inhibits generation, expansion or stimulation of regulatory T cells. In another embodiment, the agent causes a reduction in T cell anergy. The reduction in T cell anergy can be in tumor-specific T cells.

In some embodiments, a method of inhibiting abnormal cell proliferation is provided comprising administering an agent that reduces B7-H1 interaction with PD-1 in combination or alternation with a mammalian cell based vaccine and further administering an anti-cancer agent.

In some embodiments, the host in need of treatment is diagnosed with cancer. In some embodiments, the cancer is a prostate cancer. In other embodiments, the cancer is breast cancer. In other embodiments, the cancer is a renal cancer. In some embodiments, the host has been previously treated with an anti-cancer agent. In other embodiments, the host is treatment naïve.

In one embodiment, the agent reduces tolerance of T cells to a cancer. In one embodiment, the agent increases susceptibility of the cancer cell to an anti-cancer agent. In another embodiment, the agent increases susceptibility of the cancer cells to immune rejection.

### BRIEF DESCRIPTION OF THE DRAWINGS

FIG. 1 is a graph showing % survival over time in mice bearing 5 day B16 melanoma tumors were either not treated (NT, black circles), treated with GVAX vaccine (GVAX, black squares) or with a combination of GVAX and blocking anti-B7-H1 antibodies (B7H1+GVAX, open circles).

FIG. 2 is a graph showing % cancer-specific survival over time from Nephrectomy to last follow up for three years in for patients with Stage 2 and 3 renal cancers for patients in which less than 5% of cells in tissue samples stained positive for B7-H1 expression on tumor cells and infiltrating nontumor cells (B7-H1) versus patients in which greater than 5% of cells stained positive (B7-H1⁺). Calculated risk ratio: 4.53; 95% CI: 1.94-10.56; p<0.001.

FIG. 3 shows a series of histograms of CD8+ cells from a patient with chronic HCV were stained with HCV specific HLA-A2 tetrarners and anti-PD-1 antibodies.

FIG. 4 shows early blockade of PD-1/B7-H1 increases in-vivo effector cytokine production. a, Thy1.1 congenic, HA-specific CD8 T cells were adoptively transferred to indicated hosts, and harvested on day +4. Intracellular staining for IFN-γ was performed after 5 h in vitro stimulation with 1 mg/ml HA Class I Kd peptide (IYSTVASSL), in absence (top row) or presence (middle row) of PD-1 blocking antibody cocktail (30 mg/ml). n=3 animals/group b,c HA-specific CD8 T cells were adoptively transferred to c 3-HA^{low} animals as above and PD-1/B7-H1 or B7-DC blocked in vivo with 100 mg of indicated antibody administered at the time of adoptive transfer. Intracellular staining for IFNg performed on Day +6 post adoptive transfer as above. b, representative FACS plots, gated on Thy1.1-1-donor) lymphocytes. c, Summary data, mean +/− SEM. n=5, representative of 2 experiments.

FIG. 5 is a graph of % specific lysis of HA-specific CD8 T cells adoptively transferred to c3-HA^{low} animals, with indicated blocking antibodies administered I.P. on Day 0. Specific lysis was assayed by transfer of CFSE or PKH-26 labeled, HA-peptide loaded targets on Day +6. Targets from WT, B7-H1 KO and B7-DC KO animals, were differentially labeled and administered simultaneously. n=5.

FIG. 6 is a graph of % H-2K^{b}/OVA tetramer in days after antigen injection in B6 mice given OT-1 cells prior to i.v. administration of 0.5 mg OVA peptide. 10 days later, mice were given 100 mg of control hamster IgG (Cont mAb), anti-B7-H1 mAb (B7-H1 mAb) or anti-PD-1 mAb (PD-1 mAb) with (A) or without (B), 0.5 mg OVA peptide. Blood were taken from mice at the time points indicated, and the percentage of OT-1 cells present in each mouse was analyzed by FACS. An electronic gate was set on CD8⁺. Numbers refer to %- H-2K^{b}/OVA tetramer-positive cells.

### DETAILED DESCRIPTION OF THE INVENTION

It has been found that a combination of 1) an agent that blocks B7-H1 interactions with its ligand PD-1 and 2) a vaccine is synergistic in overcoming natural T cell tolerance or functional inactivation induced by tumor cells or by chronic infections. Therefore, in one embodiment, a method of enhancing efficacy of a vaccine is provided comprising administering an agent that blocks B7-H1 interactions with PD-1 in combination with the vaccine to a host in need thereof. In certain embodiments, a method of treating or preventing abnormal cell proliferation in a host is provided, comprising administering an agent that blocks B7-H1 interactions with PD-1 in combination with a vaccine against the cancer to a host in need thereof. In certain other embodiments, a method of treating chronic infection in a host is provided comprising administering an agent that blocks B7-H1 interactions with PD-1 in combination with a vaccine against the infection to a host in need thereof.

### Methods of Reducing Resistance to Antigens

In one principal embodiment, methods are provided for enhancing an immune response in a host in need thereof comprising administering an anti-B7-H1 antibody in combination with a antigen.

In another embodiment, a method of eliciting an immune response in a host is provided comprising administering an agent that interferes with B7-H1 binding to PD-1 in combination with an antigen to the host. In certain embodiments, the host is suffering from an infection. In one subembodiment, the infection is a chronic infection. In another subembodiment, the infection is an acute infection. In one embodiment, the infection is due to a virus. In another embodiment, the infection is due to a bacteria. In one embodiment, the infection is a chronic infection such as HIV, HBV, EBV or HCV.

In some principal embodiments, methods of treating or preventing an infection in a host are provided. These methods can reduce the risk of developing a chronic infection in the host. In other embodiments, the methods reduce the level of a microbe, such as a virus, in a host. In yet other embodiments, the methods reduce the infectious potential of a microbe in a host.

In one embodiment, the antigen is a viral protein. In another embodiment, the antigen is a bacterial protein. In yet another embodiment, the antigen is a mammalian protein. In certain embodiments, the antigen is expressed in a Listeria species. The Listeria species can be a Listeria monocytogenes. Methods of producing Listeria vaccines, including Listeria species expressing antigens of interest are discussed in U.S. Patent Application Publication Nos. 2004/0228877, 2005/0249748 and 2005/0281783. In certain embodiments, the Listeria species is attenuated for entry into non-phagocytic cells as compared to a wild type Listeria species. In certain cases, the Listeria species is one in which the inlB gene has been deleted (i.e., a strain attenuated for entry into non-phagocytic cells, for example, hepatocytes via the c-met receptor) or both the actA gene and the inlB genes have been deleted (i.e., a strain attenuated for both entry into non-phagocytic cells and cell-to-cell spread).

In one specific embodiment, a method of treating or preventing an infection in a host is provided comprising administering to a host in need thereof an agent that reduces B7-H1 interaction with PD-1 in combination or alternation with a cell-based vaccine. In one embodiment, the cell based vaccine is a viral cell. In certain embodiments, the vaccine is a viral cell that is not capable of infection. The virus can be irradiated. In certain embodiments, the virus is genetically modified. In other embodiments, the vaccine is not based on a cell. In certain embodiments, the vaccine is a DNA-based vaccine. In other embodiments, the vaccine is not a DNA based vaccine.

In some embodiments, the agent and vaccine can be administered in the same composition. In certain other embodiments, the agent and vaccine are administered in separate compositions. In some embodiments, the agent and vaccine are administered concurrently in separate preparations. In other embodiments, the agent is administered before administration of the vaccine. In certain embodiments, the agent is administered within one hour of the vaccine. In certain embodiments, the administration of the agent and vaccine are overlapping but not contiguous. For example, in certain embodiments, the vaccine can be administered intravenously for at least one hour and the agent may be administered orally during the intravenous administration.

In one embodiment, a composition comprising an agent that blocks B7-H1 binding to PD-1 and a vaccine is provided. In certain embodiments, the agent is an antibody and in certain specific embodiments, is an antibody that binds B7-H1. In some embodiments, the anti-B7-H1 antibody binds to the protein and changes its conformation so that B7-H1 no longer binds to PD-1. In other embodiments, the agent binds to B7-H1 at the PD-1 binding site and blocks interaction with PD-1. In some other embodiments, the agent binds to PD-1 and blocks PD-1 from interaction with B7-H1. In certain embodiments, the agent is an antibody to PD-1.

In some embodiments, the composition is an injectible composition. In certain embodiments, the composition comprises a carrier suitable for intravenous administration. In certain other embodiments, the composition comprises a carrier suitable for subcutaneous or intramuscular injection. In certain other embodiments, the composition comprises a carrier suitable for intraperitoneal administration. In other embodiment, the composition can be administered by oral administration.

In some embodiments, administration of the agent reduces tolerance of T cells to an infection with a microbe. In another embodiment, the antibody enhances an immune response against the antigen. In these embodiments, the agent that reduces B7-H1 interaction with PD-1 increases susceptibility of viruses or bacteria to immune rejection. In certain embodiments, the immune response elicited by the agent that reduces B7-H1 interaction with PD-1 is a reduction in regulatory T cells. In one embodiment, the agent enhances generation of memory T cells. In yet other embodiments, the agent inhibit generation, expansion or stimulation of regulatory T cells. In further embodiments, the agent causes a reduction in T cell anergy. The reduction in T cell anergy can be in microbe-specific T cells. In certain embodiments, the agent that reduces B7-H1 interaction with PD-1 enhances the number of antigen specific memory T cells in a host. In another embodiment, the immune response is an enhancement of effector cytokine release. In certain embodiments, this is IFN-γrelease.

In some embodiments, a method of treating or preventing an infection in a host is provided comprising administering an agent that reduces B7-H1 interaction with PD-1 in combination or alternation with a vaccine and further administering an anti-viral or anti-biotic agent.

In some embodiments, the host in need of treatment is diagnosed with a chronic infection. In some embodiments, the infection is viral. In other embodiments, the infection is bacterial. In other embodiments, the infection is HIV. In other embodiments, the infection is HCV. In some embodiments, the host has been previously treated with an antiviral agent. In other embodiments, the host is treatment naive. In one embodiment, the host is infected with the infectious agent, such as a microbe. In certain embodiments, the infectious agent is a virus. In other embodiments, the infectious agent is a bacteria. In yet other embodiments the infectious agent is a protein, such as a prion. In another embodiment, the agent increases susceptibility of a virus in the host to immune rejection.

The B7-H1 antibody can be administered at least twice, at least three times, at least four times, at least five times, at least six times, at least seven times, at least eight times, at least nine times, at least ten times or more, or between 2 and 20, between 2 and 15, between 2 and 10 or fewer times. The administration can be every day, or can be less, such as every two days, every three days, every four days, every five days, every six days, every seven days or less, such as every two weeks, once a month, once every two months, four times a year, three times a year, two times a year or once a year.

In one subembodiment, the antigen is administered less than one day after administration of the antibody. The antigen can be administered at least twice, at least three times, at least four times, at least five times, at least six times, at least seven times, at least eight times, at least nine times, at least ten times or more, or between 2 and 20, between 2 and 15, between 2 and 10 or fewer times. The administration can be every day, or can be less, such as every two days, every three days, every four days, every five days, every six days, every seven days or less, such as every two weeks, once a month, once every two months, four times a year, three times a year, two times a year or once a year.

### Methods of Treating or Preventing Abnormal Cell Proliferation

In some embodiments, a method of treating or preventing abnormal cell proliferation in a host is provided, comprising administering an agent that blocks B7-H1 interactions with PD-1 in combination with a vaccine against the cancer to a host in need thereof. These methods can reduce the risk of developing cancer in the host. In other embodiments, the methods reduce the amount of cancer in a host. In yet other embodiments, the methods reduce the metastatic potential of a cancer in a host. The methods can also reduce the size of a cancer in a host.

In one embodiment, the agent that blocks B7-H1 binding to PD-1 is an antibody. In certain embodiments, the agent is an antibody that binds to B7-H1 and inhibits its interaction with PD-1. In certain embodiments, the agent is an agent that binds to B7-H1 and changes its conformation so that the protein no longer binds to PD-1. In other embodiments, the agent binds to B7-H1 at the PD-1 binding site and blocks interaction with PD-1. In some other embodiments, the agent binds to PD-1 and blocks PD-1 from interaction with B7-H1. In certain embodiments, the agent is an antibody to PD-1.

In some embodiments, the agent and vaccine can be administered in the same composition. In certain other embodiments, the agent and vaccine are administered in separate compositions. In some embodiments, the agent and vaccine are administered concurrently in separate preparations. In other embodiments, the agent is administered before administration of the vaccine. In certain embodiments, the agent is administered within one hour of the vaccine. in certain embodiments, the administration of the agent and vaccine are overlapping but not contiguous. For example, in certain embodiments, the vaccine can be administered intravenously for at least one hour and the agent may be administered orally during the intravenous administration.

In one embodiment, the agent and cell based vaccine are administered in combination. In certain of these embodiments, the agent and vaccine are administered concurrently in the same preparation. In other embodiments, the agent and vaccine are administered concurrently in separate preparations. In other embodiments, the agent is administered before administration of the vaccine. In some embodiments, the vaccine is administered at least one hour, at least 8 hours, 1 day or 2 days after administration of the agent. In certain embodiments, the agent and vaccine arc administered in multiple rounds. In specific embodiments, the agent and vaccine are administered at least 2, at least 3, at least 4, at least 5, at least 6, at least 7, at least 8 at least 9 or at least 10 times.

In some embodiments, the method further comprises administering an anti-cancer agent in the absence of the agent. In some embodiments, the anti-cancer agent is administered at least 1, at least 2, at least 3, at least 4, at least 5, at least 6, at least 7, at least 8 at least 9 or at least 10 days, or at least 1 week, a least 2 weeks, at least 3 weeks, at least 1 month, at least 2 months, at least 3 months, at least 4 months, at least 5 months, at least 6 months or more after administration of the vaccine.

In one embodiment, a composition comprising an agent that blocks B7-H1 binding to PD-1 and a vaccine is provided. In certain embodiments, the agent is an antibody and in certain specific embodiments, is an antibody that binds B7-H1. In some embodiments, the anti-B7-H1 antibody binds to the protein and changes its conformation so that B7-H1 no longer binds to PD-1.

In some embodiments, the composition is an injectible composition. In certain embodiments, the composition comprises a carrier suitable for intravenous administration. In certain other embodiments, the composition comprises a carrier suitable for subcutaneous or intramuscular injection. In certain other embodiments, the composition comprises a carrier suitable for intraperitoneal administration. In other embodiment, the composition can be administered by oral administration.

In some embodiments, administration of the agent reduces tolerance of T cells to a cancer. In these embodiments, the agent that reduces B7-H1 interaction with PD-1 increases susceptibility of cancer cells to immune rejection. In certain embodiments, the immune response elicited by the agent that reduces B7-H1 interaction with PD-1 is a reduction in regulatory T cells. In yet other embodiments, the agent inhibit generation, expansion or stimulation of regulatory T cells. In further embodiments, the agent causes a reduction in T cell anergy. The reduction in T cell anergy can be in tumor-specific T cells.

In one specific embodiment, a method of treating or preventing abnormal cell proliferation in a host is provided comprising administering to a host in need thereof an agent that reduces B7-H1 interaction with PD-1 in combination or alternation with a mammalian cell based vaccine.

In one embodiment, the mammalian cell based vaccine is a whole mammalian cell. In certain embodiments, the vaccine is a tumor cell that is not actively dividing. The tumor cell can be irradiated. In certain embodiments, the cell is genetically modified. In some embodiments, the cell can be secreting an activation factor for an antigen-presenting cell. In certain embodiments, the cell secretes, for example constitutively secretes, a colony stimulating factor and can specifically secrete a granulocyte-macrophage colony stimulating factor (GM-CSF). The cell can be based on cells from the same type of tissue as the tumor. In certain embodiments, the cell is derived from a prostate cancer cell. In other embodiments, the cell is derived from a breast cancer cell. In other instances, the cell is derived from a lymphoma cell.

In one embodiment, the agent that reduces B7-H1 interaction with PD-1 reduces tolerance of T cells to a cell in the cell based vaccine. In this embodiment, the agent increases susceptibility of tumor cells to immune rejection. In one embodiment, the immune response is a reduction in regulatory T cells. In one embodiment, the agent enhances generation of memory T cells. In yet another embodiment, the agent inhibits generation, expansion or stimulation of regulatory T cells. In another embodiment, the agent causes a reduction in T cell anergy. The reduction in T cell anergy can be in tumor-specific T cells.

In some embodiments, a method of inhibiting abnormal cell proliferation is provided comprising administering an agent that reduces B7-H1 interaction with PD-1 in combination or alternation with a mammalian cell based vaccine and further administering an anti-cancer agent.

In some embodiments, the host in need of treatment is diagnosed with cancer. In some embodiments, the cancer is a prostate cancer. In other embodiments, the cancer is breast cancer. In other embodiments, the cancer is a renal cancer. In some embodiments, the host has been previously treated with an anti-cancer agent. In other embodiments, the host is treatment naive.

In one embodiment, the agent reduces tolerance of T cells to a cancer. In one embodiment, the agent increases susceptibility of the cancer cell to an anti-cancer agent. In another embodiment, the agent increases susceptibility of the cancer cells to immune rejection.

In another principal embodiment, a method of treating or preventing abnormal cell proliferation is provided comprising administering an agent reduces B7-H1 interaction with PD-1 to a host in need thereof in combination with an antigen and substantially in the absence of an anti-cancer agent.

In one embodiment, the first agent stimulates an immune response for at least one day. In another embodiment, the agent stimulates an immune response for at least one week.

The agent reduces B7-H1 interaction with PD-1 can be administered at least twice, at least three times, at least 4 times, at least 5 times, at least 6 times, at least 7 times, at least 8 times, at least 9 times, at least 10 times or more, or between 2 and 20, between 2 and 15, between 2 and 10 or fewer times. The administration can be every day, or can be less often, such as every two days, every three days, every four days, every five days, every six days, every seven days or less, such as every two weeks, once a month, once every two months, four times a year, three times a year, two times a year or once a year.

In one embodiment, the agent reduces tolerance of T cells to a cancer. In one embodiment, the agent increases susceptibility of the cancer cell to an anti-cancer agent. In another embodiment, the agent increases susceptibility of the cancer cells to immune rejection.

### B7-H1 Monoclonal Antibodies

Methods of making antibodies are known in the art. For example, they can be produced by immunizing animals with a substance of interest (e.g., B7-H1). A useful antibody can be a polyclonal antibody present in the serum or plasma of an animal (e.g., human, non-human primate, mouse, rabbit, rat, guinea pig, sheep, horse, goat, cow, pig, or bird) which has been injected with the substance of interest, and optionally an adjuvant. Polyclonal and monoclonal antibodies can be manufactured in large amounts by methods known in the art.

Polyclonal antibodies can be isolated from serum or plasma by methods known in the art. For example, large animals (e.g., sheep, pigs, goats, horses, or cows) or a large number of small animals can be immunized as described above. Serum can be isolated from the blood of animals producing an antibody with the appropriate activity. If desired, polyclonal antibodies can be purified from such sera by methods known in the art.

Monoclonal antibodies (mAb) can also be produced. Methods of making and screening monoclonal antibodies arc well known in the art. Once the desired antibody producing hybridoma has been selected and cloned, the resultant antibody can be produced by a number of methods known in the art. For example, the hybridoma can be cultured in vitro in a suitable medium for a suitable length of time, followed by the recovery of the desired antibody from the supernatant. The length of time and medium are known or can be readily determined. Monoclonal antibodies can also be produced in large amounts in vitro using, for example, bioreactors or in vivo by injecting appropriate animals with the relevant hybridoma cells. For example, mice or rats can be injected intraperitoneally (i.p.) with the hybridoma cells and, after a time sufficient to allow substantial growth of the hybridoma cells and secretion of the monoclonal antibody into the blood of the animals, they can be bled and the blood used as a source of the monoclonal antibody. If the animals are injected i.p. with an inflammatory substance such as pristane and the hybridoma cells, peritoneal exudates containing the monoclonal antibodies can develop in the animals. The peritoneal exudates can then be “tapped” from the animals and used as a source of the appropriate monoclonal antibody.

### Co-stimulatory Molecules

In addition to antigen-specific signals mediated through the T-cell receptor, T cells also require antigen nonspecific costimulation for activation. The B7 family of molecules on antigen-presenting cells, which include B7-1 (CD80) and B7-2 (CD86), play important roles in providing costimulatory signals required for development of antigen-specific immune responses. The CD28 molecule on T cells delivers a costimulatory signal upon engaging either of its ligands, B7.1 (CD80) or B7.2 (CD86) and possibly B7.3. A distinct signal is transduced by the CD40L (for ligand) molecule on the T cell when it is ligated to CD40. A number of other molecules on the surface of APC may serve some role in costimulation, although their full role or mechanism of action is not clear. These include VCAM-1, ICAM-1 and LFA-3 on APC and their respective ligands VLA-4, LFA-1 and CD2 on T cells. It is likely that the integrins LFA-1 and VCAM-1 are involved in initiating cell-cell contact. LFA-1 (lymphocyte function associated protein 1) which blocks killing of target cells by CD8 cytotoxic T cells. LFA-1 binds the immunoglobulin superfamily ligands ICAM-1, -2, -3. Blocking β-2 integrin is a very effective way of inhibiting immune responses and monoclonal antibodies against this protein are in clinical trial for treatment of transplant recipients and other conditions. Other immunotherapeutics in development are CTLA-Ig, which is a soluble from of a high affinity receptor for B7.1 and B7.2 (more avid than CD28), and anti-CD40L. Both block co-stimulation of T cells and anti-CD40L may also block reciprocal activation of antigen presenting cells.

In some embodiments, the agent that blocks B7-H1 binding to PD-1 is administered in combination or alternation with an agent that activates a CD28 pathway. In certain instances, this costimmulatory molecule is a B7.1 or B7-2 or B7-3 molecule. In certain instances, the costimmulatory molecule is a B7-DC or B7-H1 molecule, and in particular a protein fusion of B7-DC, B7-H1, variants of these or truncates thereof. In specific embodiments, the costimmulatory molecule is an Fc-fusion of a B7-H1 or B7-DC molecule, a fragment of a B7-H1 or B7-DC molecule, or a variant thereof. In certain cases, the variant can include one or more mutated amino acids when compared to the native protein. In certain embodiments, the costimmulatory molecule does not interact with PD-1. In other embodiments, the agent that blocks B7-H1 binding to PD-1 is administered in combination or alternation with an antibody that blocks interaction of soluble B7-H4 with its ligand. In certain embodiments, the costimulatory molecule is encoded by a vector derived from a virus. For example a costimmulatory molecule can be encoded by a vector derived from a canarypox virus, ALVAC. In some embodiments, the costimmulatory molecule is B7.1, encoded by a vector derived from the canarypox virus, ALVAC (ALVAC-B7.1), alone or with another molecule, such as interleukin 12 (ALVAC-IL-12).

Checkpoint inhibitors can also be used in conjunction with the agent that blocks B7-H1 binding to PD-1 of the invention. For example, inhibitors of PD-1 could be used to reduce inhibition of T cell activity. In addition, molecules such as soluble B7-H4 can be used to stimulate T cell activities.

In certain embodiments, the agent that reduces B7-H1 interaction with PD-1 is administered in combination or alternation with a specific human antibody. The specific antibody generally acts as a passive vaccine, providing immediate immunity against certain agents. The antibody can be directed against agents such as anthrax, toxins produced by Clostridium botulinum, Brucellosis, Q fever (caused by Coxiella burnetii), smallpox, viral meningoencephalitis syndromes (including Eastern equine encephalomyelitis virus (EEEV), Venezuelan equine encephalomyelitis virus (VEEV), and Western equine encephalomyelitis virus (WEEV)), viral hemorrhagic fevers (including Ebola, Marburg, and Junin), tularemia, biological toxins (including those causing diphtheria, tetanus, botulism, venoms, ricin, trichothecene mycotoxins, and staphylococcal enterotoxins) and plague.

### Anti-Cancer Agents

In certain embodiments, the methods of the invention are provided in combination with an anti-cancer agent to treat abnormal cell proliferation. Many of these drugs can be divided in to several categories: alkylating agents, antimetabolites, anthracyclines, plant alkaloids, topoisomerase inhibitors, monoclonal antibodies, and other antitumour agents. Some agents don't directly interfere with DNA. These include the new tyrosine kinase inhibitor imatinib mesylate (Gleevec® or Glivec®), which directly targets a molecular abnormality in certain types of cancer (chronic myelogenous leukemia, gastrointestinal stromal tumors).

Alkylating agents are so named because of their ability to add alkyl groups to many electronegative groups under conditions present in cells. Cisplatin and carboplatin, as well as oxaliplatin are alkylating agents. Other agents are mechloethamine, cyclophosphamide, chlorambucil. They work by chemically modifying a cell's DNA.

Anti-metabolites masquerade as purine ((azathioprine, mercaptopurine)) or pyrimidine—which become the building blocks of DNA. They prevent these substances becoming incorporated in to DNA during the “S” phase (of the cell cycle), stopping normal development and division. They also affect RNA synthesis. Due to their efficiency, these drugs are the most widely used cytostatics.

Plant alkaloids and terpenoids are derived from plants and block cell division by preventing microtubule function. Microtubules are vital for cell division and without them it can not occur. The main examples are vinca alkaloids and taxanes. Vinca alkaloids bind to specific sites on tubulin, inhibiting the assembly of tubulin into microtubules (M phase of the cell cycle). They are derived from the Madagascar periwinkle, Catharanthus roseus (formerly known as Vinca rosea). The vinca alkaloids include: Vincristine; Vinblastine; Vinorelbine; and Vindesine. Podophyllotoxin is a plant-derived compound used to produce two other cytostatic drugs, etoposide and teniposide. They prevent the cell from entering the G1 phase (the start of DNA replication) and the replication of DNA (the S phase). The substance has been primarily obtained from the American Mayapple (Podophyllum peltatum). Recently it has been discovered that a rare Himalayan Mayapple (Podophyllum hexandrum) contains it in a much greater quantity, but as the plant is endangered, its supply is limited. Taxanes are derived from the Yew Tree. Paclitaxel (Taxol®) is derived from the bark of the Pacific Yew Tree (Taxus brevifolia). Researchers had found a much renewable source, where the precursors of Paclitaxel can be found in relatively high amounts in the leaves of the European Yew Tree (Taxus baccata), and that Paclitaxel, and Docetaxel (a semi-synthetic analogue of Paclitaxel) could be obtained by semi-synthetic conversion. Taxanes enhance stability of microtubules, preventing the separation of chromosomes during anaphase. Taxanes include: Paclitaxel and Docetaxel.

Topoisomerase inhibitors are another class of compounds. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins: irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophiyllum peltatum).

Antitumour antibiotics are another class of anti-cancer compounds. The most important immunosuppressant from this group is dactinomycin, which is used in kidney transplantations. Monoclonal antibodies work by targeting tumour specific antigens, thus enhancing the host's immune response to tumour cells to which the agent attaches itself. Examples are trastuzumab (Herceptin), cetuximab, and rituximab (Rituxan or Mabthera). Bevacizumab is a monoclonal antibody that does not directly attack tumor cells but instead blocks the formation of new tumor vessels.

Several malignancies are also potentially treated with hormonal therapy. Steroids (often dexamethasone) can inhibit tumour growth or the associated edema (tissue swelling), and may cause regression of lymph node malignancies. Prostate cancer is often sensitive to finasteride, an agent that blocks the peripheral conversion of testosterone to dihydrotestosterone. Breast cancer cells often highly express the estrogen and/or progesterone receptor. Inhibiting the production (with aromatase inhibitors) or action (with tamoxifen) of these hormones can often be used as an adjunct to therapy. Gonadotropin-releasing hormone agonists (GnRH), such as goserelin possess a paradoxic negative feedback effect followed by inhibition of the release of FSH (follicle-stimulating hormone) and LH (luteinizing hormone), when given continuously.

General examples of anti-cancer agents also include: ifosamide, cisplatin, methotrexate, cytoxan, procarizine, etoposide, BCNU, vincristine, vinblastine, cyclophosphamide, gencitabine, 5-flurouracil, paclitaxel, and doxorubicin. Additional agents that are used to reduce cell proliferation include: AS-101 (Wyeth-Ayers″ Labs.), bropirimine (Upjohn), gamma interferon (Genentech), GM-CSF (Genetics Institute), IL-2 (Cetus or Hoffman-LaRoche), human immune globulin (Cutter Biological), 20 IMREG (from Imreg of New Orleans, La.), SKF106528 (Genentech), TNF (Genentech), azathioprine, cyclophosphamide, chlorambucil, and methotrexate.

### Antigen/Infections

In one embodiment of the invention, the method provides an enhanced and prolonged immune response to an antigen. An antigen is generally any compound, composition, or agent, as well as all related antigenic epitopes, capable of being the target of inducing a specific immune response, such as stimulate the production of antibodies or a T-cell response in a subject, including compositions that are injected or absorbed into a subject. In some embodiments, the host is infected with a virus or bacteria that has an antigen prior to the administration of the agent that blocks B7-H1 binding to PD-1.

For example, the host can be infected with an HIV virus. In other embodiments, the host is infected with a flavivirus or pestivirus, or other member of the flaviviridae family such as hepatitis C. Pestiviruses and flaviviruses belong to the flaviviridae family of viruses along with hepacivirus (hepatitis C virus). The pestivirus genus includes bovine viral diarrhea virus (BVDV), classical swine fever virus (CSFV, also called hog cholera virus) and border disease virus (BDV) of sheep (Moennig, V. et al. Adv. Vir. Res. 1992, 41, 53-98). Pestivirus infections of domesticated livestock (cattle, pigs and sheep) cause significant economic losses worldwide. BVDV causes mucosal disease in cattle and is of significant economic importance to the livestock industry (Meyers, G. and Thiel, H.-J., Advances in Virus Research, 1996, 47, 53-118; Moennig V., et al, Adv. Vir. Res. 1992, 41, 53-98). In certain embodiments, the host is infected with a hepatitis B virus. In other embodiments, the host is infected with hepatitis D (also known as hepatitis delta). In certain embodiments, the host is infected with a member of the herpes family, such as Herpes simplex virus, Cytomegalovirus, and Epstein-Barr virus (EBV).

Antigens can include: live, heat killed, or chemically attenuated viruses, bacteria, mycoplasmas, fungi, and protozoa, or fragments, extracts, subunits, metabolites and recombinant constructs of these or fragments, subunits, metabolites and recombinant constructs of mammalian proteins and glycoproteins; nucleic acids; combinations of these; or whole mammalian cells.

Antigens can be from pathogenic and non-pathogenic organisms, viruses, and fungi. Antigens can include proteins, peptides, antigens and vaccines from smallpox, yellow fever, distemper, cholera, fowl pox, scarlet fever, diphtheria, tetanus, whooping cough, influenza, rabies, mumps, measles, foot and mouth disease, and poliomyelitis.

The antigen can be a protein or peptide. In certain embodiments, the antigen is exogenous. The antigen can, for example, be a viral or bacterial protein or peptide, or antigenic fragment thereof. In certain instances, the antigen is from a “subunit” vaccine, composed of viral or bacterial antigenic determinants, generally in which viral or bacterial antigens made are free of nucleic acid by chemical extraction and containing only minimal amounts of non-viral or non-bacterial antigens derived from the culture medium. In other instances, the antigen is not based on a subunit vaccine.

Peptide epitopes can also be derived from any of a variety of infectious microorganisms. Peptide epitopes can be expressed on any relevant cells need not be classical APC but can be any cell infected with an appropriate infectious microorganism. Such cells include, without limitation, T cells, tissue epithelial cells, endothelial cells, and fibroblasts. Thus, the methods of the invention can be applied to the treatment of infections by any of a wide variety of infectious microorganisms. While such microorganisms will generally be those that replicate inside a cell (commonly designated intracellular pathogens), the methods of the invention can also be applied to situations involving infectious microorganisms that replicate extracellularly or in cells that do not express B7-H1. Relevant microorganisms can be viruses, bacteria, mycoplasma, fungi (including yeasts), and protozoan parasites and specific examples of such microorganisms include, without limitation, Mycobactevia tubevculosis, Salmonella enteviditis, Listevia monocytogenes, M. lepvae, Staphylococcus auveus, Eschevichia coli, Slveptococcuspneumoniae, Bovvelia buvgdorfevi, Actinobacillus pleuvopneumoniae, Helicobactev pylovi, Neissevia meningitidis, Yevsinia entevocolitica, Bovdetella pertussis, Povphyvomonas gingivalis, mycoplasma, Histoplasma capsulatum, Cvyptococcus neofovmam, Chlamydia tvachomatis, Candida albicans, Plasmodium falcipavum, Entamoeba histolytica, Toxoplasma bvucei, Toxoplasma gondii, Leishmania major human immunodeficiency virus 1 and 2, influenza virus, measles virus, rabies virus, hepatitis virus A, B, and C, rotaviruses, papilloma virus, respiratory syncytial virus, feline immunodeficiency virus, feline leukemia virus, and simian immunodeficiency virus.

In certain embodiments, the antigen is a whole cell, derived from a virus, bacteria or mammal. In certain embodiments, the antigen is a “killed component” of a vaccine. In some embodiments of the invention, the antigen is derived from a human or animal pathogen. The pathogen is optionally a virus, bacterium, fungus, or a protozoan. In this instance, the antigen is prepared from a viral or bacterial cell that has been irradiated or otherwise inactivated to avoid replication. In one embodiment, the antigen is a protein produced by the pathogen, or a fragment and/or variant of a protein produced by the pathogen. In other embodiments, the antigen is a mammalian protein or peptide. In certain embodiment, the antigen is a whole mammalian cell and is not an isolated mammalian protein or peptide, or fragment thereof.

In some embodiments, the antigen is a whole cell. In some embodiments, the antigen is a whole mammalian cell, which can be genetically modified. In certain embodiments, the cell is a whole mammalian tumor cell that has been modified to express a colony stimulating factor. In other embodiments, the antigen is a stromal antigen-presenting cell capable of antigen presentation.

In some embodiments, the antigen may be derived from Human Immunodeficiency virus (such as gp120, gp 160, gp41, gag antigens such as p24gag and p55gag, as well as proteins derived from the pol, env, tat, vif, rev, nef, vpr, vpu and LTR regions of HIV), Feline Immunodeficiency virus, or human or animal herpes viruses. In one embodiment, the antigen is derived from herpes simplex virus (HSV) types 1 and 2 (such as gD, gB, gH, Immediate Early protein such as ICP27), from cytomegalovirus (such as gB and gH), from Epstein-Barr virus or from Varicella Zoster Virus (such as gpI, II or III). (See, e.g., Chee et al. (1990) Cytomegaloviruses (J. K. McDougall, ed., Springer Verlag, pp. 125-169; McGeoch et al. (1988) J. Gen. Virol. 69: 1531-1574; U.S. Pat. No. 5,171,568; Baer et al. (1984) Nature 310: 207-211; and Davison et al. (1986) J. Gen. Virol. 67: 1759-1816.)

In another embodiment, the antigen is derived from a hepatitis virus such as hepatitis B virus (for example, Hepatitis B Surface antigen), hepatitis A virus, hepatitis C virus, delta hepatitis virus, hepatitis E virus, or hepatitis G virus. See, e.g., WO 89/04669; WO 90/11089; and WO 90/14436. The hepatitis antigen can be a surface, core, or other associated antigen. The HCV genome encodes several viral proteins, including E1 and E2. See, e.g., Houghton et al., Hepatology 14: 381-388(1991).

An antigen that is a viral antigen is optionally derived from a virus from any one of the families Picornaviridae (e.g., polioviruses, rhinoviruses, etc.); Caliciviridae; Togaviridae (e.g., rubella virus, dengue virus, etc.); Flaviviridae; Coronaviridae, Reoviridae (e.g., rotavirus, etc.); Birnaviridae; Rhabodoviridae (e.g., rabies virus, etc.); Orthomyxoviridae (e.g., influenza virus types A, B and C, etc.); Filoviridae; Paramyxoviridae (e.g., mumps virus, measles virus, respiratory syncytial virus, parainfluenza virus, etc.); Bunyaviridae; Arenaviridae; Retroviradae (e.g., HTLV-I; HTLV-11; HIV-1; HIVI11b; HIVSF2; HTVLAV; HIVLAI; HIVMN; HIV-1CM235; HIV-2; simian immunodeficiency virus (SIV)); Papillomavirus, the tick-borne encephalitis viruses; and the like. See, e.g. Virology, 3rd Edition (W. K. Joklik ed. 1988); Fundamental Virology, 3rd Edition (B. N. Fields, D. M. Knipe, and P. M. Howley, Eds. 1996), for a description of these and other viruses. In one embodiment, the antigen is Flu-HA (Morgan et al., J. Immunol. 160:643 (1998)).

In one embodiment, the antigen comprises a (Myco)bacterial or viral protein or an immunogenic part, derivative and/or analogue thereof. In one aspect of the invention, the antigen comprises a Mycobacterium protein or an immunogenic part, derivative and/or analogue thereof. In one embodiment, the antigen comprises hsp65 369 412 (Ottenhof et al., 1991; Charo et al., 2001). In another embodiment, the antigen comprises a human papillomavirus (HPV) protein or an immunogenic part, derivative and/or analogue thereof. An immunogenic part, derivative and/or analogue of a protein comprises the same immunogenic capacity in kind not necessarily in amount as said protein itself. A derivative of such a protein can be obtained by conservative amino acid substitution. In one embodiment, the antigen is a killed whole pneumococci, lysate of pneumococci or isolated and purified PspA, or immunogenic fragments thereof (see U.S. Pat. No. 6,042,838). In one embodiment, the antigen is a 314 amino acid truncate (amino acids 1-314) of the mature PspA molecule. This region of the PspA molecule contains most, if not all, of the protective epitopes of PspA.

In some embodiments, the antigen is derived from bacterial pathogens such as Mycobacterium, Bacillus, Yersinia, Salmonella, Neisseria, Borrelia (for example, OspA or OspB or derivatives thereof), Chlamydia, or Bordetella (for example, P.69, PT and FHA), or derived from parasites such as plasmodium or Toxoplasma. In one embodiment, the antigen is derived from the Mycobacterium tuberculosis (e.g. ESAT-6, 85A, 85B, 72F), Bacillus anthracis (e.g. PA), or Yersinia pestis (e.g. F1, V). In addition, antigens suitable for use in the present invention can be obtained or derived from known causative agents responsible for diseases including, but not limited to, Diptheria, Pertussis, Tetanus, Tuberculosis, Bacterial or Fungal Pneumonia, Otitis Media, Gonorrhea, Cholera, Typhoid, Meningitis, Mononucleosis, Plague, Shigellosis or Salmonellosis, Legionaire's Disease, Lyme Disease, Leprosy, Malaria, Hookworm, Onchocerciasis, Schistosomiasis, Trypamasomialsis, Lesmaniasis, Giardia, Amoebiasis, Filariasis, Borelia, and Trichinosis. Still further antigens can be obtained or derived from unconventional pathogens such as the causative agents of kuru, Creutzfeldt-Jakob disease (CJD), scrapie, transmissible mink encephalopathy, and chronic wasting diseases, or from proteinaceous infectious particles such as prions that are associated with mad cow disease.

A large number of tumor-associated antigens that are recognized by T cells have been identified (Renkvist et al., Cancer Immunol Innumother 50:3-15 (2001)). These tumor-associated antigens may be differentiation antigens (e.g., PSMA, Tyrosinase, gp100), tissue-specific antigens (e.g. PAP, PSA), developmental antigens, tumor-associated viral antigens (e.g. HPV 16 E7), cancer-testis antigens (e.g. MAGE, BAGE, NY-ESO-1), embryonic antigens (e.g. CEA, alpha-fetoprotein), oncoprotein antigens (e.g. Ras, p53), over-expressed protein antigens (e.g. ErbB2 (Her2/Neu), MUC1), or mutated protein antigens.

Tumor-associated antigens that may be useful in the methods of the invention include, but are not limited to, 707-AP, Annexin II, AFP, ART-4, BAGE, β-catenin/m, BCL-2, bcr-abl, bcr-abl p190, bcr-abl p210, BRCA-1, BRCA-2, CAMEL, CAP-1, CASP-8, CDC27/m, CDK-4/m, CEA (Huang et al., Exper Rev. Vaccines (2002)1:49-63), CT9, CT10, Cyp-B, Dek-cain, DAM-6 (MAGE-B2), DAM-10 (MAGE-B1), EphA2 (Zantek et al., Cell Growth Differ. (1999) 10:629-38; Carles-Kinch et al., Cancer Res. (2002) 62:2840-7), ELF2M, ETV6-AML1, G250, GAGE-1, GAGE-2, GAGE-3, GAGE-4, GAGE-5, GAGE-6, GAGE-7B, GAGE-8, GnT-V, gp100, HAGE, HER2/neu, HLA-A*0201-R170I, HPV-E7, HSP70-2M, HST-2, hTERT, hTRT, iCE, inhibitors of apoptosis (e.g. survivin), KIAA0205, K-ras, LAGE, LAGE-1, LDLR/FUT, MAGE-1, MAGE-2, MAGE-3, MAGE-6, MAGE-A1, MAGE-A2, MAGE-A3, MAGE-A4, MAGE-A6, MAGE-A10, MAGE-A12, MAGE-B5, MAGE-B6, MAGE-C2, MAGE-C3, MAGE-D, MART-1, MART-1/Melan-A, MC1R, MDM-2, mesothelin, Myosin/m, MUC1, MUC2, MUM-1, MUM-2, MUM-3, neo-polyA polymerase, NA88-A, NY-ESO-1, NY-ESO-1a (CAG-3), PAGE-4, PAP, Proteinase 3 (Molldrem et al., Blood (1996) 88:2450-7; Molldrem et al., Blood (1997) 90:2529-34), P15, p190, Pm1/RARα, PRAME, PSA, PSM, PSMA, RAGE, RAS, RCAS1, RU1, RU2, SAGE, SART-1, SART-2, SART-3, SP 17, SPAS-1, TEL/AML 1, TPI/m, Tyrosinase, TARP, TRP-1 (gp75), TRP-2, TRP-2/INT2, WT-1, and alternatively translated NY-ESO-ORF2 and CAMEL proteins.

In some embodiments, the antigen that is not identical to a tumor-associated antigen, but rather is derived from a tumor-associated antigen. For instance, the antigen may comprise a fragment of a tumor-associated antigen, a variant of a tumor-associated antigen, or a fragment of a variant of a tumor-associated antigen. In some cases, an antigen, such as a tumor antigen, is capable of inducing a more significant immune response when the sequence differs from that endogenous to the host. In some embodiments, the variant of a tumor-associated antigen, or a fragment of a variant of a tumor-associated antigen, differs from that of the tumor-associated antigen, or its corresponding fragment, by one or more amino acids. The antigen derived from a tumor-associated antigen can comprise at least one epitope sequence capable of inducing an immune response upon administration.

Alternatively, the antigen can be an autoimmune disease-specific antigen. In a T cell mediated autoimmune disease, a T cell response to self antigens results in the autoimmune disease. The type of antigen for use in treating an autoimmune disease with the vaccines of the present invention might target the specific T cells responsible for the autoimmune response. For example, the antigen may be part of a T cell receptor, the idiotype, specific to those T cells causing an autoimmune response, wherein the antigen incorporated into a vaccine of the invention would elicit an immune response specific to those T cells causing the autoimmune response. Eliminating those T cells would be the therapeutic mechanism to alleviating the autoimmune disease. Another possibility would be to incorporate an antigen that will result in an immune response targeting the antibodies that are generated to self antigens in an autoimmune disease or targeting the specific B cell clones that secrete the antibodies. For example, an idiotype antigen may be incorporated into the Listeria that will result in an anti-idiotype immune response to such B cells and/or the antibodies reacting with self antigens in an autoimmune disease.

In still other embodiments, the antigen is obtained or derived from a biological agent involved in the onset or progression of neurodegenerative diseases (such as Alzheimer's disease), metabolic diseases (such as Type I diabetes), and drug addictions (such as nicotine addiction). Alternatively, the method can be used for pain management and the antigen is a pain receptor or other agent involved in the transmission of pain signals.

### Diseases and Disorders of Abnormal Cell Proliferation

In certain embodiments, the present invention can be used to treat or prevent cancer as well as other abnormal cell proliferation-associated diseases in a host. A host is any multi-cellular vertebrate organism including both human and non-human mammals. In one embodiment, the “host” is a human. The terms “subject” and “patient” are also included in the term “host”.

In certain embodiments, the present invention provides methods to treat carcinomas, include tumors arising from epithelial tissue, such as glands, breast, skin, and linings of the urogenital, digestive, and respiratory systems. Lung, cancer and prostate cancers can be treated or prevented. Breast cancers that can be treated or prevented include both invasive (e.g., infiltrating ductal carcinoma, infiltrating lobular carcinoma infiltrating ductal & lobular carcinoma, medullary carcinoma, mucinous (colloid) carcinoma, comedocarcinoma, paget's disease, papillary carcinoma, tubular carcinoma, adenocarcinoma (NOS) and carcinoma (NOS)) and non-invasive carcinomas (e.g., intraductal carcinoma, lobular carcinoma in situ (LCIS), intraductal & LCIS, papillary carcinoma, comedocarcinoma). The present invention can also be used to treat or prevent metastatic breast cancer. Non-limiting examples of metastatic breast cancer include bone, lung and liver cancer.

Prostate cancers that can be treated or prevented with the methods described herein include localized, regional and metastatic prostate cancer. Localized prostate cancers include A1-A2, T1a-T1b, T1c, B0-B2 or T2a-T2c. C1-C2 or T3a-N0, prostate cancers extending beyond the prostate but without lymph node involvement, are also contemplated. Regional prostate cancers include D1 or N1-M0, while metastatic prostate cancers include D2 or M1. Metastatic prostate cancers include bone and brain cancers.

In certain embodiments, methods are provided to treat or prevent abnormal cell proliferation using agent that blocks B7-H1 binding to PD-1 in combination or alternation with a cell based vaccine. In certain of these embodiments, the cell based vaccine is based on cells that match the tumor to be prevented. For example, if a host is suffering from, or at risk of suffering from, a prostate cancer, the cell based vaccine will be based on a prostate cancer tumor cell. In these instances, the cell is typically irradiated or otherwise prevented from replicating. In particular embodiments, the cell is genetically modified to secrete a colony stimulating factor.

Other cancers that can be treated or prevented with the present invention include, but are not limited to, cancers of the cancers include those of the bowel, bladder, brain, cervix, colon, rectum, esophagus, eye, head and neck, liver, kidney, larynx, lung, skin, ovary, pancreas, pituitary gland, stomach, testicles, thymus, thyroid, uterus, and vagina as well as adrenocortical cancer, carcinoid tumors, endocrine cancers, endometrial cancer, gastric cancer, gestational trophoblastic tumors, islet cell cancer, and mesothelioma.

Lymphomas that can be treated or prevented with the invention include tumors arising from the lymph or spleen, which can cause excessive production of lymphocytes, including both Hodgkin's disease and Non- Non-Hodgkin's lymphoma. The term “Hodgkin's Disease” is intended to include diseases classified as such by the REAL and World Health Organization (WHO) classifications known to those of skill in the art, including classical Hodgkin's disease (i.e., nodular sclerosis, mixed cellularity, lymphocyte depletion or lymphocyte rich) or lymphocyte predominance Hodgkin's disease. The term “Non-Hodgkin's lymphoma” is used to refer 30 lymphomas classified by WHO (Harris N L, Jaffe E S, Kiebold J, Flandrin G, Muller-Hermelink H K, Vardiman J. Lymphoma classification-from controversy to consensus: the REAL and WHO Classification of lymphoid neoplasms. Ann Oncol. 2000;11 (suppl 1):S3-S10), including but not limited to:

B-cell non-Hodgkin's lymphomas such as small lymphocytic lymphoma (SLL/CLL), mantle cell lymphoma (MCL), follicular lymphoma marginal zone lymphoma (MZL), extranodal (MALT lymphoma), nodal (Monocytoid B-cell lymphoma), splenic, diffuse large cell lymphoma, burkitt's lymphoma and lymphoblastic lymphoma.

T-cell non-Hodgkin's lymphoma's such as lymphoblastic lymphomas, peripheral T-cell lymphoma. Hepatosplenic gamma-delta T-cell lymphoma, subcutaneous panniculitis-like lymphoma, angioimmunoblastic T-cell lymphoma (AILD), extranodal NK/T cell lymphoma, nasal type, intestinal T-cell lymphoma (+/− enteropathy associated) (EATL), adult T-cell leukemia/lymphoma (HTLV-1 associated), mycosis fungoides/Sezary syndrome, anaplastic large cell lymphoma (ALCL), including both primary cuteous and primary systemic types.

Leukemias that can be treated or prevented with the present invention include but are not limited to myeloid and lymphocytic (sometimes referred to as B or T cell leukemias) or myeloid leukemias, both chronic and acute. The myeloid leukemias include chronic myeloid leukemia (CML) and acute myeloid leukemia (AML) (i.e., acute nonlymphocytic leukemia (ANLL)). The lymphocytic leukemias include acute lymphocytic leukemia (ALL), chronic lymphocytic leukemia (CLL) (i.e., chronic granulocytic leukemia) and hairy cell leukemia (CCL).

Sarcomas that can be treated or prevented with the present invention include both bone and soft-tissue sarcomas of the muscles, tendons, fibrous tissues, fat, blood vessels nerves, and synovial tissues. Non-limiting examples include fibrosacromas, rhabdomyosarcomas, liposarcomas, synovial sarcomas, angiosacromas, neurofibrosarcomas, gastrointestinal stroma tumors, Kaposi's sacroma, Ewing's sarcoma, alveolar soft-part sarcoma, angiosarcoma, dermatofibrosarcoma protuberans, epithelioid sarcoma, extraskeletal chondrosarcoma, extraskeletal osteosarcoma, fibrosarcoma, leiomyosarcoma, liposarcoma, malignant fibrous histiocytoma, malignant hemangiopericytoma, malignant mesenchymoma, malignant schwannoma, malignant peripheral nerve sheath tumor, parosteal osteosarcoma, peripheral neuroectodermal tumors, rhabdomyosarcoma, synovial sarcoma, and sarcoma, NOS.

Diseases of abnormal cell proliferation other than cancer can be treated or prevented with the present invention. Diseases association with the abnormal proliferation of vascular smooth muscle cells include, as a non-limiting example, benign tumors. Non-limiting examples of benign tumors include benign bone, brain and liver tumors.

Other diseases associated with abnormal cell proliferation include, for example, atherosclerosis and restenosis. Diseases associated with abnormal proliferation of over-proliferation and accumulation of tissue mast cells are also included, such as cutaneous mastocytosis (CM) and Urticaria pigmentosa. Diseases associated with abnormal proliferation of xesangial cell proliferation are also contemplated, including but not limited to IgA nephropathy, membranoproliferative glomerulonephritis (GN), lupus nephritis and diabetic nephropathy.

Psoriasis can be treated or prevented by the present invention, including but not limited to, plaque psoriasis, guttate psoriasis, inverse psoriasis, seborrheic psoriasis, nail psoriasis, generalized erythrodermic psoriasis (also called psoriatic exfoliative erythroderm), pustular psoriasis, and Von Zumbusch psoriasis.

The present invention can also be used to treat or prevent lymphangiomyomatosis (LAM), as well as other diseases associated with abnormal cell proliferation known to those skilled in the art.

### Pharmaceutical Compositions

The described compounds can be formulated as pharmaceutical compositions and administered for any of the disorders described herein, in a host, including a human, in any of a variety of forms adapted to the chosen route of administration, including systemically, such as orally, or parenterally, by intravenous, intramuscular, topical, transdermal or subcutaneous routes.

The compounds can be included in the pharmaceutically acceptable carrier or diluent in an amount sufficient to deliver to a patient a therapeutically effective amount to treat cancer or other disorders characterized by abnormal cell proliferation or cancer or the symptoms thereof in vivo without causing serious toxic effects in the patient treated.

A dose of the agent that blocks B7-H1 binding to PD-1 for the above-mentioned conditions will be in the range from about 1 to 75 mg/kg, or 1 to 20 mg/kg, of body weight per day, more generally 0.1 to about 100 mg per kilogram body weight of the recipient per day. The effective dosage range of the prodrug can be calculated based on the weight of the parent derivative to be delivered.

The compounds are conveniently administered in units of any suitable dosage form, including but not limited to one containing 7 to 3000 mg, or 70 to 1400 mg of active ingredient per unit dosage form. An oral dosage of 50-1000 mg is usually convenient, and more typically, 50-500 mg.

In certain instances, the agent that blocks B7-H1 binding to PD-1 should be administered to achieve peak plasma concentrations of the active compound of from about 0.2 to 70 μM, or about 1.0 to 10 μM. This may be achieved, for example, by the intravenous injection of an appropriate concentration of the active ingredient, optionally in saline, or administered as a bolus of the active ingredient.

The concentration of the agent that blocks B7-H1 binding to PD-1 in the drug composition will depend on absorption, inactivation and excretion rates of the extract as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The agent that blocks B7-H1 binding to PD-1 may be administered at once, or may be divided into a number of smaller doses to be administered at varying intervals of time.

One mode of administration of the agent that blocks B7-H1 binding to PD-1 is oral. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The agent that blocks B7-H1 binding to PD-1 can be administered as a component of an elixir, suspension, syrup, wafer, chewing gum or the like. A syrup may contain, in addition to the active compounds, sucrose as a sweetening agent and certain preservatives, dyes and colorings and flavors. The compounds can also be mixed with other active materials that do not impair the desired action, or with materials that supplement the desired action, such as antibiotics, antifungals, anti-inflammatories, or other anti-autoimmune compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In another embodiment, the compounds are prepared with carriers that will protect the derivatives against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art.

Liposomal suspensions (including liposomes targeted to infected cells with monoclonal antibodies to viral antigens) are also typical as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension.

In some embodiments, the agent that blocks B7-H1 binding to PD-1 can be administered in a composition that enhances the half life of the agent that blocks B7-H1 binding to PD-1 in the body. For example, the agent that blocks B7-H1 binding to PD-1 can be linked to a molecule, such as a polyethylene glycol. In certain embodiments, the molecule can be used to target the agent that blocks B7-H1 binding to PD-1 to a cell, for example as a ligand to a receptor. In some embodiments, the linking of the agent that blocks B7-H1 binding to PD-1 reduces the amount of times the agent that blocks B7-H1 binding to PD-1 is administered in a day or in a week. In other embodiments, the linkage can enhance the oral availability of the agent that blocks B7-H1 binding to PD-1.

In certain instances, the compositions will additionally comprise an immunogenic adjuvant. Antigens, especially when recombinantly produced, may elicit a stronger response when administered in conjunction with adjuvant. Alum is an adjuvant licensed for human use and hundreds of experimental adjuvants such as cholera toxin B are being tested. Helicobacter pylori is the spiral bacterium which selectively colonizes human gastric mucin-secreting cells and is the causative agent in most cases of nonerosive, gastritis in humans. Recent research activity indicates that H. pylori, which has a high urease activity, is responsible for most peptic ulcers as well as many gastric cancers. Many studies have suggested that urease, a complex of the products of the ureA and ureB genes, may be a protective antigen.

Immunogenicity can be significantly improved if an antigen is co-administered with an adjuvant, commonly used as 0.001% to 50% solution in phosphate buffered saline (PBS). Adjuvants enhance the immunogenicity of an antigen but are not necessarily immunogenic themselves. Intrinsic adjuvants, such as lipopolysaccarides, normally are the components of the killed or attenuated bacteria used as vaccines. Extrinsic adjuvants are immunomodulators which are typically non-covalently linked to antigens and are formulated to enhance the host immune response. Aluminum hydroxide and aluminum phosphate (collectively commonly referred to as alum) are routinely used as adjuvants in human and veterinary vaccines. A wide range of extrinsic adjuvants can provoke potent immune responses to antigens. These include saponins complexed to membrane protein antigens (immune stimulating complexes), pluronic polymers with mineral oil, killed mycobacteria in mineral oil, Freund's complete adjuvant, bacterial products, such as muramyl dipeptide (MDP) and lipopolysaccharide (LPS), as well as lipid A, and liposomes. To efficiently induce humoral immune response (HIR) and cell-mediated immunity (CMI), immunogens are typically emulsified in adjuvants.

U.S. Pat. No. 4,855,283 granted to Lockhoff describes glycolipid analogs including N-glycosylamides, N-glycosylureas and N-glycosylcarbamates, each of which is substituted in the sugar residue by an amino acid, as immune-modulators or adjuvants. U.S. Pat. No. 4,258,029 granted to Moloney describes that octadecyl tyrosine hydrochloride (OTH) functions as an adjuvant when complexed with tetanus toxoid and formalin inactivated type I, II and III poliomyelitis virus vaccine. Octodecyl esters of aromatic amino acids complexed with a recombinant hepatitis B surface antigen, enhanced the host immune responses against hepatitis B virus. Bessler et al., “Synthetic lipopeptides as novel adjuvants,” in the 44th Forum In Immunology (1992) at page 548 et seq. is directed to employing lipopeptides as adjuvants when given in combination with an antigen. The lipopeptides typically had P3C as the lipidated moiety and up to only 5 amino acids, e.g., P3C-SG, P3C-SK4, P3C-SS, P3C-SSNA, P3C-SSNA.

Antigens or immunogenic fragments thereof stimulate an immune response when administered to a host. In one embodiment, the antigen is a killed whole pneumococci, lysate of pneumococci or isolated and purified PspA, as well as immunogenic fragments thereof, particularly when administered with an adjuvant (see U.S. Pat. No. 6,042,838). The S. pneumoniae cell surface protein PspA has been demonstrated to be a virulence factor and a protective antigen (see WO 92/14488). In an effort to develop a vaccine or immunogenic composition based on PspA, PspA has been recombinantly expressed in E. coli. It has been found that in order to efficiently express PspA, it is useful to truncate the mature PspA molecule of the Rx1 strain from its normal length of 589 amino acids to that of 314 amino acids comprising amino acids 1 to 314. This region of the PspA molecule contains most, if not all, of the protective epitopes of PspA. It would be useful to improve the immunogenicity of recombinant PspA and fragments thereof. Moreover, it would be highly desirable to employ a pneumococcal antigen in a combination or multivalent composition.

Nardelli et al. (Vaccine (1994), 12(14):1335 1339) covalently linked a tetravalent multiple antigen peptide containing a gp120 sequence to a lipid moiety and orally administered the resulting synthetic lipopeptide to mice. Croft et al. (J. Immunol. (1991), 146(5): 793 796) have covalently coupled integral membrane proteins (Imps) isolated from E. coli to various antigens and obtained enhanced immune responses by intramuscular injection into mice and rabbits. Schlecht et al. (Zbl. Bakt. (1989) 271:493 500) relates to Salmonella typhimurium vaccines supplemented with synthetically prepared derivatives of a bacterial lipoprotein having five amino acids. Substantial effort has been directed toward the development of a vaccine for Lyme disease.

### Dosing

The compounds are generally administered for a sufficient time period to alleviate the undesired symptoms and the clinical signs associated with the condition being treated. In one embodiment, the compounds are administered less than three times daily. In one embodiment, the compounds are administered in one or two doses daily. In one embodiment, the compounds are administered once daily. In some embodiments, the compounds are administered in a single oral dosage once a day. In certain embodiments, as described above, the antibody is administered in a specific order and in a particular time frame, to provide the tolerance inducing effects and reduce the use of immunosuppressive agents.

The active compound is included in the pharmaceutically acceptable carrier or diluent in an amount sufficient to deliver to a patient a therapeutic amount of compound in vivo in the absence of serious toxic effects. An effective dose can be determined by the use of conventional techniques and by observing results obtained under analogous circumstances. In determining the effective dose, a number of factors are considered including, but not limited to: the species of patient; its size, age, and general health; the specific disease involved; the degree of involvement or the severity of the disease; the response of the individual patient; the particular compound administered; the mode of administration; the bioavailability characteristics of the preparation administered; the dose regimen selected; and the use of concomitant medication.

Typical systemic dosages for the herein described conditions are those ranging from 0.01 mg/kg to 1500 mg/kg of body weight per day as a single daily dose or divided daily doses. Dosages for the described conditions typically range from 0.5-1500 mg per day. A more particularly dosage for the desired conditions ranges from 5-750 mg per day. Typical dosages can also range from 0.01 to 1500, 0.02 to 1000, 0.2 to 500, 0.02 to 200, 0.05 to 100, 0.05 to 50, 0.075 to 50, 0.1 to 50, 0.5 to 50, 1 to 50, 2 to 50, 5 to 50, 10 to 50,25 to 50,25 to 75,25 to 100, 100 to 150, or 150 or more mg/kg/day, as a single daily dose or divided daily doses. In one embodiment, the daily dose is between 10 and 500 mg/day. In another embodiment, the dose is between about 10 and 400 mg/day, or between about 10 and 300 mg/day, or between about 20 and 300 mg/day, or between about 30 and 300 mg/day, or between about 40 and 300 mg/day, or between about 50 and 300 mg/day, or between about 60 and 300 mg/day, or between about 70 and 300 mg/day, or between about 80 and 300 mg/day, or between about 90 and 300 mg/day, or between about 100 and 300 mg/day, or about 200 mg/day. In one embodiment, the compounds are given in doses of between about 1 to about 5, about 5 to about 10, about 10 to about 25 or about 25 to about 50 mg/kg. Typical dosages for topical application are those ranging from 0.001 to 100% by weight of the active compound.

The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only.

### EXAMPLES

### Example 1

### Anti-B7-H1 Antibodies and Vaccine Produce Synergistic Reactions

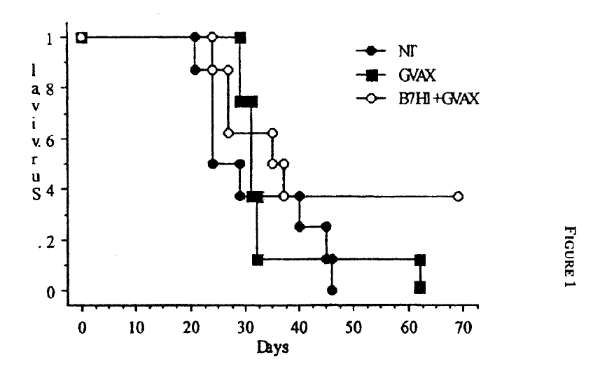

Synergy was shown between a GM-CSF transduced vaccine (GVAX) and anti-B7-H1 antibodies in the treatment of B16 melanoma. Hybridoma cell lines producing antibody anti-B7-H1 is deposited as ______. Transduction of B7-H1-tumors with the B7-H1 gene results in surface expression of B7-H1 with resultant protection from elimination by a tumor vaccine. Likewise, blocking antibodies to B7-H1 will enhance the capacity of T cells to eliminate tumors that naturally express B7-H1. Blocking anti-B7-H1 antibodies were combined with vaccination using GM-CSF transduced tumor vaccines (GVAX). Mice bearing 5 day B16 melanoma tumors were either not treated, treated with GVAX vaccine or with a combination of GVAX+blocking anti-B7H1 antibodies. Results are demonstrated in FIG. 1, which shows synergy between the GVAX vaccine and the antibodies. The combination resulted in 40% longterm survival.

### Example 2

### Expression of B7-H1 on Human Renal Cancer Correlates with Poor Prognosis

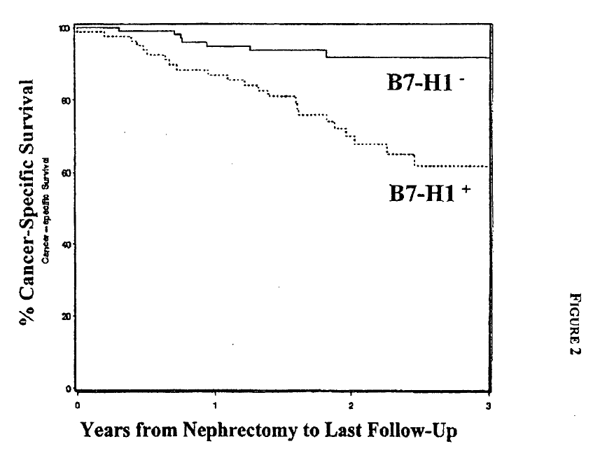

A recent clinical study compared the expression of B7-H1 in human renal cancers with survival. In a retrospective analysis, tissue samples from surgically resected Stage 2 and 3 renal cancers were stained for expression of B7-H1 on tumor cells and infiltrating nontumor cells. <5% positive cells were categorized as negative and >5% positive cells were categorized as positive. Long term cancer-specific survival was analyzed for the two groups. This study demonstrated a dramatic correlation between expression of B7-H1 on both tumor cells and infiltrating cells within the tumor and poor prognosis. The results are shown in FIG. 2. These clinical results strongly suggest that B7-H1 expression on human cancers as well as induced B7-H1 expression on infiltrating cells protects the tumor from immune attack, thereby favoring the tumor.

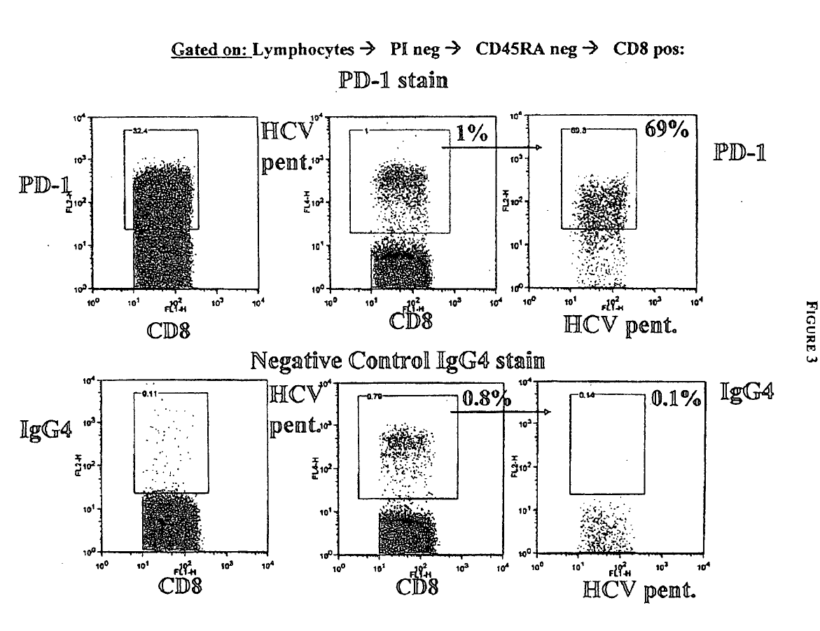

HCV specific T cells from a patient with chronic HCV express elevated levels of PD-1. Because the liver is known to express high levels of B7-H1, it is likely that the PD-1 expressing T cells are inhibited from eliminating HCV-infected hepatocytes due to inhibition by B7-H1/PD-1 interactions. These interactions will also inhibit the activity of T cells induced by HCV vaccines, potentially explaining why no therapeutic HCV vaccine has ever cleared HCV in primate models. Anti-human B7-H1 antibodies were produced that amplify human T cell responses in vitro. CD8+ cells from a patient with chronic HCV were stained with HCV specific HLA-A2 tetrarners and anti-PD-1 antibodies. The majority of HCV specific CD8 T cells express high levels of PD-1 (FIG. 3).

### Example 3

### Early blockade of PD-1/B7-H1 Increases in-vivo Effector Cytokine Production and Reverses Functional Tolerance in vivo

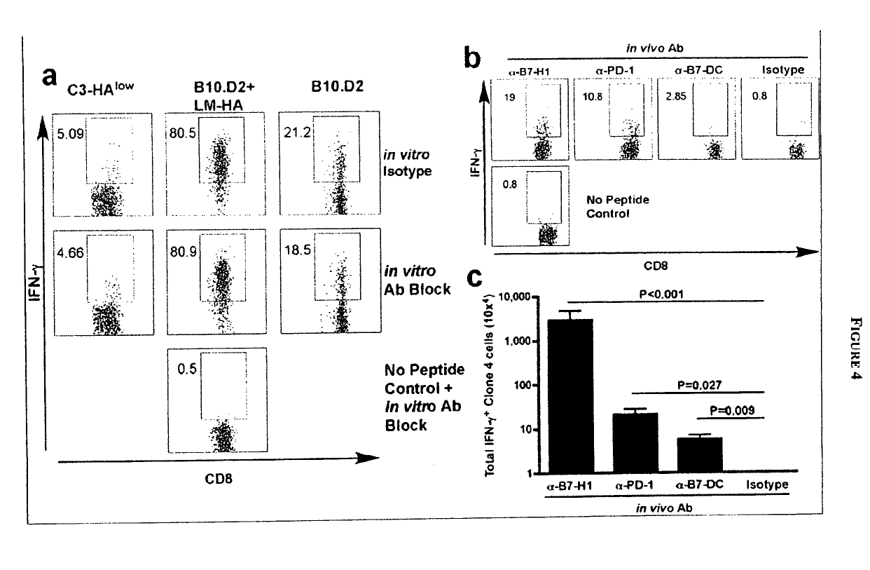

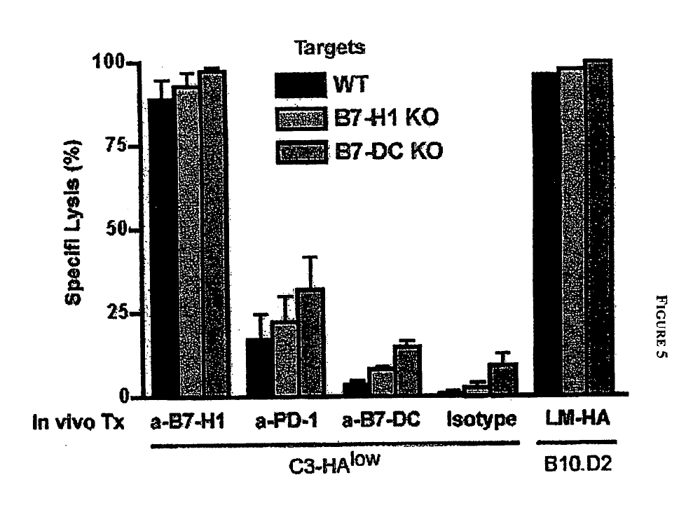

One role of the B7-H1/PD1 interaction is in the initial decision that T cells make to become tolerant vs activated. FIGS. 4 and 5 demonstrate that blocking B7-H1 or PD1 with antibodies at the time of transfer of naive antigen-specific CD8 T cells into an animal where the antigen is expressed as a self antigen results in activation rather than tolerance induction as measured by IFN-γ production and in vivo CTL activity.

Thy1.1 congenic, HA-specific CD8 T cells were adoptively transferred to hosts and harvested on day +4. Intracellular staining for IFN-g was performed after 5 h in vitro stimulation with 1 mg/ml HA Class I Kd peptide (IYSTVASSL) in the absence or presence of a PD-1 blocking antibody cocktail (30 mgl ml). Separately, HA-specific CD8 T cells were adoptively transferred to c3-HA^{low} animals and PD-1/B7-H1 or B7-DC blocked in vivo with 100 mg of antibody administered i.p. at the time of adoptive transfer. Intracellular staining for LFNγ performed on Day +6 post adoptive transfer. Separately, specific lysis by T cells was assayed by transfer of CFSE or PKH-26 labeled, HA-peptide loaded targets on Day +6. Targets from WT, B7-H1 KO and B7-DC KO animals, were differentially labeled (see methods) and administered simultaneously.

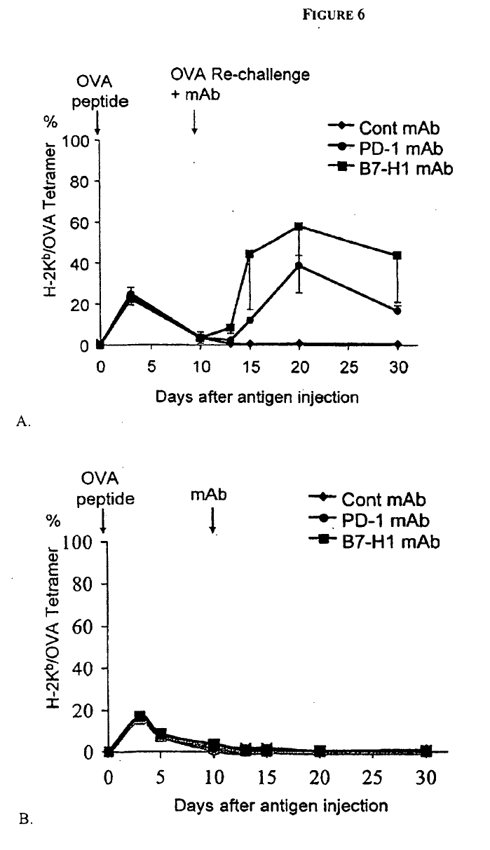

It should be noted that that antibodies to B7-H1 have a much more potent effect than antibodies to PD1. Furthermore, a peptide immunization together with anti-B7-H1 antibodies can REVERSE the inactivated state of tolerant T cells and result in activated effector T cells. These results are shown in FIG. 6a. B6 mice were given OT-1 cells prior to i.v. administration of 0.5 mg OVA peptide. Ten days later, mice were given 100 mg of control hamster IgG, anti-B7-H1 mAb, anti-B7-DC mAb or anti-PD-1 mAb with or without 0.5 mg OVA peptide. Blood were taken from mice and the percentage of OT-1 cells present in each mouse was analyzed by FACS.

This reversal of tolerance is dependent on both the peptide vaccination and anti-B7-H1 administration, since anti-B7-H1 without peptide vaccination failed to reverse tolerance. This result further demonstrates that the combination of vaccine and anti-B7-H1 antibody is critical for synergy in tolerance reversal.

1. A method of enhancing efficacy of a vaccine comprising administering an agent that blocks B7-H1 interactions with PD-1 in combination with the vaccine to a host in need thereof.

2. A method of treating or preventing abnormal cell proliferation in a host comprising administering an agent that blocks B7-H1 interactions with PD-1 in combination with a vaccine against the cancer to a host in need thereof.

3. The method of claim 2 wherein the vaccine is a mammalian cell based vaccine.

4. The method of claim 3 wherein the mammalian cell based vaccine is a whole mammalian cell.

5. The method of claim 4 wherein the mammalian cell secretes a granulocyte-macrophage colony stimulating factor (GM-CSF).

6. The method of claim 2 further comprising administering an anti-cancer agent.

7. The method of claim 2 wherein the host has been diagnosed with cancer.

8. The method of claim 1 or 2 wherein the agent that blocks B7-H1 binding to PD-1 is an antibody.

9. The method of claim 8 wherein the antibody binds to B7-H1 and inhibits its interaction with PD-1.

10. A method of treating chronic infection in a host comprising administering an agent that blocks B7-H1 interactions with PD-1 in combination with an antigen to a host in need thereof.

11. The method of claim 10 wherein the agent that blocks B7-H1 binding to PD-1 is an antibody.

12. The method of claim 11 wherein the antibody binds to B7-H1 and inhibits its interaction with PD-1.

13. The method of claim 10 wherein the host is suffering from a chronic infection.

14. The method of claim 13 wherein the infection is due to a virus.

15. A composition comprising an agent that blocks B7-H1 binding to PD-1 and a vaccine, optionally in a pharmaceutically acceptable carrier.

16. The composition of claim 15 wherein the agent that blocks B7-H1 binding to PD-1 is an antibody.

17. The composition of claim 15 wherein the composition is an suitable for intravenous injection.
